(19) United States
(12) Patent Application Publication
(10) Pub. No.: US 2023/0258539 A1
(43) Pub. Date: Aug. 17, 2023

(54) SWAB WITH RECEPTACLE PROVIDING INTEGRATED DRYING

(71) Applicant: Weavr Health Corp., Cambridge, MA (US)

(72) Inventors: Brandon T. JOHNSON, Cambridge, MA (US); Kate E. CHRISTIAN, Baltimore, MD (US)

(21) Appl. No.: 17/933,466

(22) Filed: Sep. 19, 2022

Related U.S. Application Data
(63) Continuation of application No. PCT/US2021/022907, filed on Sep. 23, 2021.
(60) Provisional application No. 62/991,864, filed on Mar. 19, 2020.
(60) Provisional application No. 63/018,171, filed on Apr. 30, 2020.

(51) Int. Cl. G01N 1/10

(52) U.S. Cl. CPC G01N 1/10; G01N 2001/1056

(57) ABSTRACT

Described herein is a device for collecting and drying and protecting oral or nasal swab samples that can then be submitted for antibody and/or molecular testing.

### CROSS-REFERENCE TO RELATED APPLICATIONS

This application is a continuation of International Application No. PCT/US2021/022907, filed on Sep. 23, 2021, which claims the benefit of U.S. Provisional Application No. 63/018,171, filed Apr. 30, 2020 and U.S. Provisional Patent Application No. 62/991,864, filed Mar. 19, 2020, each of which is hereby incorporated by reference in its entirety for all purposes.

### BACKGROUND

Influenza is highly contagious, viral infection of the respiratory tract caused by immunologically diverse, single-strand RNA viruses known as influenza viruses. Influenza viruses are transmitted primarily by the inhalation of airborne contaminated micro droplets from other infected individuals and influenza can be epidemic in proportions infecting many individuals at the same time.

Early diagnosis is critical in prescribing the correct medications and recuperative treatment for the disease.

Some have developed kits useful for extracting viral antigens. One example is the QuickVue (R) Influenza Test available from Craig Medical Distribution, Inc. of Vista, Calif. A swab containing a nasal discharge specimen is placed in a special extraction tube with a supplied test reagent solution during which time the virus particles in the specimen are disrupted, exposing internal viral nucleoproteins. The Influenza Test Strip is then placed in the extracted reagent solution where nucleoproteins in the specimen react with the impregnated reagents. If the extracted specimen contains influenza antigens, a pink-to-red Test Line along with a blue procedural Control Line will appear on the Test Strip indicating a positive result. If influenza type A or type B antigens are not present or are present at very low levels, only a blue procedural Control Line will appear.

Polymerase Chain Reaction (PCR) is a laboratory method used for making a very large number of copies of short sections of DNA from a very small sample of genetic material. This process is called “amplifying” the DNA and it enables specific genes of interest to be detected or measured. Reverse Transcriptase PCR (RT-PCR), is a variation of the polymerase chain reaction that typically measures RNA expression levels. In RT-PCR, complementary DNA (cDNA) is made by reverse transcribing of the RNA templates with the enzyme reverse transcriptase. This technique is particularly useful in identifying die presence of influenza or novel coronavirus, which are an RNA viruses. Real-time reverse transcription polymerase chain reaction (rRT-PCR) amplifies a specific target sequence in the sample, then monitors the amplification progress using fluorescent technology. As the number of DNA amplification copies increases during the PCR reaction, there is also an increase in the fluorescence. This offers the distinct advantage of obtaining real time monitoring of the PCR reaction and allows for quantitative analysis of the DNA expression.

The COVID-19 RT-PCR test is a real-time reverse transcription polymerase chain reaction (rRT-PCR) test for the qualitative detection of nucleic acid from SARS-CoV-2 in respiratory specimens collected from individuals suspected of COVID-19. These specimens are typically nasopharyngeal or oropharyngeal swabs in viral transport media but may also include sputum, lower respiratory tract aspirates, bronchoalveolar lavage, and nasopharyngeal wash/aspirate or nasal aspirate. These samples are only stable for 48 hours when refrigerated and are unstable at room temperature unless dried.

### SUMMARY

### Problem Statement

Current testing for viruses such as COVID-19/SARS/CoV-2 is unavailable to the majority of Americans due to the lack of home collection and home testing options. Diagnostic assays are currently run with nasal and oral swab samples that are only stable for 48 hours when. stored at 4 degrees centigrade, and these samples are not stable at room temperature.

In order to enable home collection of nasal and oral swab samples products need to be able to collect, treat and dry the sample while requiring no training on the part of the user. The current testing methods and venues put patients and healthcare workers alike at undue risk by requiring infected and uninfected people alike to visit testing locations, giving the virus a greater opportunity to spread. In particular this unnecessary exposure of healthcare workers to infection can compound the danger of the pandemic by effectively reducing our work force in the healthcare sector.

It has been realized that existing blood sample collection technologies such as those described in U.S. Pat. No. 8,021,873 entitled “Portable, point-of-care, user-initiated fluidic assay methods and systems”; U.S. Pat. No. 9,861,978 entitled “Mechanically actuated vacuum controlled fluid collection”; U.S. Patent Publication 2019/0111421A1 entitled “Fluid sample collection device”; and PCT Publication WO2019/231837A1 entitled “Blood metering device with desiccant and support for storage media and inlay with flange” can provide a starting point for a small, portable, easy to use device adapted for use in viral diagnostics. The referenced patents and patent publications are hereby incorporated in their entirety.

Such technologies can, for example, be used or adapted to produce products to collect and stabilize both nasal and oral swab samples as well as blood samples to be submitted to viral testing regimes based on RNA analysis. They can also be used to create a blood based home test providing results in 15 minutes as well. Home collection and testing can allow for home screening while enabling consumers and patients to remain quarantined.

More particularly, all biological samples are inherently unstable at room temperature requiring complex treatment and processing to prevent sample degradation during transport. Accurate lab results are only possible when samples arrive intact and properly preserved.

### Brief Description

Described herein is a device for collecting and drying oral or nasal swab samples that can then be submitted for antibody and/or molecular testing. The device's internal systems collect, dry and store various sample types to ensure that samples arrive intact at the lab and are ready for testing. The devices are simple enough such that the public at large can collect or even test themselves at home using the device.

In one embodiment, and from a general perspective, the device may be provided as two component parts, a housing component and a cap component that fits securely onto the housing. The housing includes a swab element, a tube connected to the swab element at one end, and to the interior body of the device at the other end. The housing also defines a drying region in which is disposed a drying agent. The cap component encloses a liquid reagent, and a retention element, such as pierceable membrane, to releasably secure the reagent inside the cap.

In use, the exposed swab element of the housing is first used to collect a sample such as nasal sample, saliva, blood or serum sample, or other body fluid. The cap is then snapped, screwed, or otherwise securely attached to the housing over the swab. The action of attaching the cap ruptures the pierceable membrane, and thus swab is then introduced to the liquid reagent which may or may not contain stabilizing reagents.

There are several possible use cases.

Antibody testing: The device can be used to collect and stabilize samples for antibody testing, This method of testing uses blood, serum or plasma to detect the body's immune response to the infection. Such tests can be run on saliva or similar samples to detect a current or past infection using immunoassays.

Molecular testing: The device can be used to collect and stabilize samples for molecular testing. This method of testing uses saliva, oral fluid, or a similar sample to detect RNA from the virus itself. This type of test can also be run on blood but current results are poor in blood samples.

Active infection is detected using Polymerase Chain Reaction (PCR). All assays currently cleared/approved/authorized to diagnose are molecular.

Pandemic response: The COVID-19 pandemic provides three distinct opportunities for use of this technology:
1. Separating, drying and transporting plasma for antibody testing.
2. Home testing for SARS-CoV-2 using lateral flow strips.
3. Preserving RNA from oral and nasal swabs for transport when collected at home.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a view of a housing component (also referred to herein as a receptacle) and cap component.

FIG. 2 is a more detailed view of the housing, taken from a direction that shows a cross-section of a paper element and a plan view of desiccant tablets.

FIG. 3 is a detailed view of another embodiment taken from the same direction as in FIG. 2, with this embodiment having multiple stacked paper elements.

FIG. 4 is a detailed view of the embodiment of FIG. 3, taken from a direction perpendicular to the view of FIG. 3, showing a cross-section of the desiccant tablets and a plan view of one of the paper elements.

FIG. 5 is an embodiment where a reagent is encased in a screw-on type cap and the drying region is disposed along one side of a receptacle for the swab.

FIG. 6 is another embodiment with a screw-on cap but having the drying region disposed on an end of the receptacle.

FIG. 7 is yet another configuration where the reagent and drying region are enclosed in the cap.

### DETAILED DESCRIPTION OF PREFERRED EMBODIMENTS

Described herein is a device for collecting and drying oral or nasal swab samples that can then be submitted for antibody and/or molecular testing.

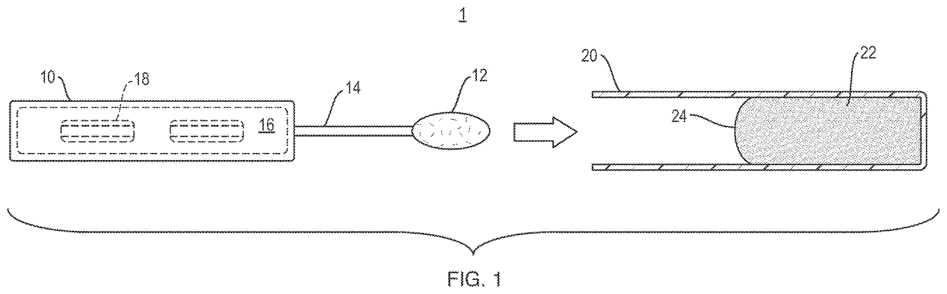

Referring to FIG. 1, generally speaking, the device 1 consists of two component parts, a housing and 10 a cap 12 that fits securely onto the housing 10.

The housing 10 includes: a swab element 12, a tube 14 connected to the swab element 12 at one end, and to the interior body of the device at the other end; a drying region 16; a drying agent 18; and with optional vent openings 13 to ambient.

The cap 20 includes: a liquid reagent 22 a retention element 24, such as pierceable membrane, to releasably seal the reagent inside.

In use, the exposed swab element 12 of the housing 10 is first used to collect the sample. The cap 20 is then snapped, screwed, or otherwise securely attached to the housing 10 over the swab 12. The action of attaching the cap ruptures the pierceable membrane, and thus swab is then introduced to the liquid reagent which may or may not contain stabilizing reagents.

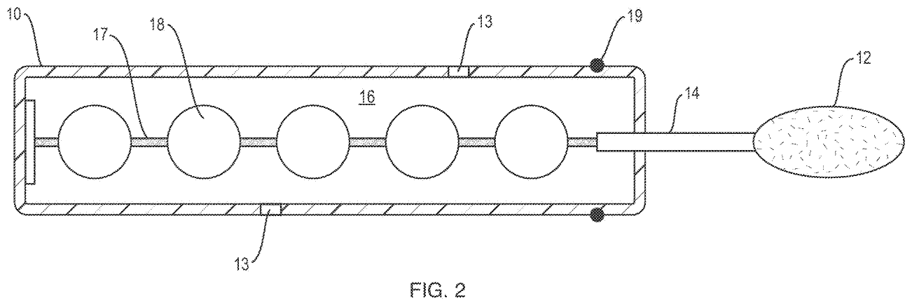

Referring now also to FIG. 2, the liquid sample is then drawn into the tube 14 from the swab 12 end and transferred to the drying region 16 at the tube exit end. A surface in the drying region may contain a paper (or membrane) 17. The sample liquid may be spread by such paper or membrane into a thin layer to facilitate faster drying. The drying agent 18, also disposed within the housing 10, may be a desiccant such as one or more desiccant tablets. The drying region 16 may be exposed to the air through openings or vent holes 13 in the housing.

The swab 12 and tube 14 may be integrally formed with the housing 10 at the time of manufacture. However, the swab and tube 14 may be separately manufactured from the housing 10 (with its enclosed drying region 16 and agent 18); with the swab inserted and retained within or onto the housing after the nasal or other fluid sample is taken.

An o-ring 19 formed of resilient material may be disposed on the exterior of the housing 10 to encourage a tight seal with the cap 12.

The paper 17 may be retained in position within the housing by a frame, inlay, substrate, adhesive or other support element or feature (not shown) as described in the above-referenced Patent Publication WO2019/231837A1 entitled “Blood metering device with desiccant and support for storage media and inlay with flange”.

Likewise, desiccant tablets 18 may similarly retained in position within the housing by a frame, substrate, adhesive or in other ways.

The device, including the housing 10 and cap 12 may be sealed in a packaging, after manufacture but before use, the packaging designed to have low moisture transfer rates.

The device, including the housing 10 and the cap 20, may be cylindrical in cross-section.

The device may require shaking to mix the sample and liquid reagent in addition or in place of flow.

Flow of the collected sample to the drying region 16 may be encouraged by gravity, by standing the device on end, such that the end opposite the cap is on the bottom.

The device may have another liquid reagent sealed in a second portion of the device such as either the cap or the housing. This second liquid reagent may also be secured via a breakable seal that it breaks when the swab element is introduced

The tube 14 connecting the swab element 12 to the drying region 16 may contain or be composed of a material to wick or otherwise facilitate flow of fluid from the swab element and liquid reagent to the drying area.

Another embodiment of the device 1 may cause a pressure differential when the cap is placed over the swab element forcing fluid into the tube and drying region and subsequently onto the paper, membrane or other drying surface.

Another embodiment of the device 1 may have a saliva receptor instead of a swab element. This saliva receptor may have a cap with liquid reagents in it. This embodiment may need to be shaken to mix the sample with the reagent thoroughly,

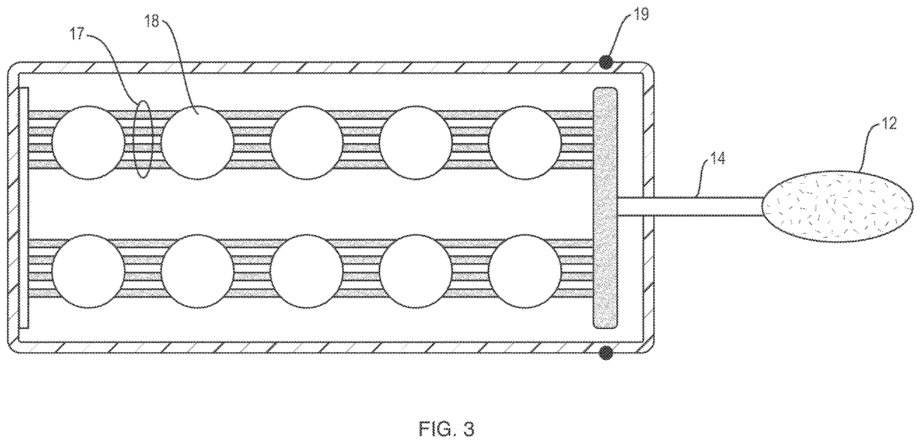

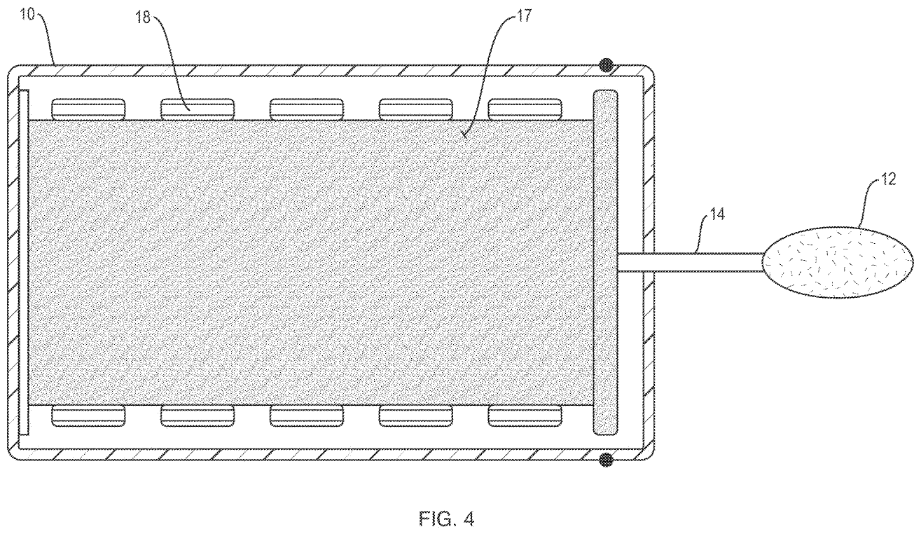

Referring to FIGS. 3 and 4, the device may include multiple membranes or treated. papers 17, with a space between them to enable drying of each one, The spacing may be provided by a frame or other support element (not shown), or by interspersing other trans missive, non-retentive media sheets between the membranes.

Although two rows of desiccant tablets are shown in the embodiment of FIGS. 3 and 4, there may only be a single such row or a single desiccant, and the tablets may be smaller or larger in diameter than what is shown.

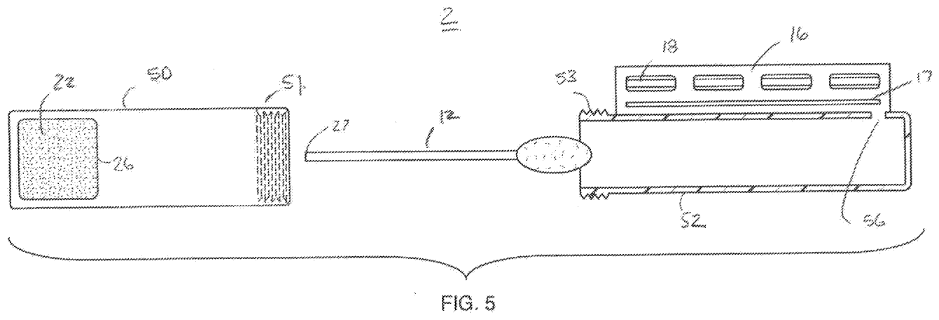

FIG. 5 is another configuration for a device 2 which provides advantages similar to those described above. The device 2 consists of screw-on type cap 50 and receptacle 52 which have corresponding threaded portions 51, 53. Here, the reagent 22 is encased via a seal or in a pouch 26 in the cap 50 and the drying region 16 is disposed along one side of the receptacle 52. The drying region 16 is in fluid communication with the receptacle such as via an opening 56.

Here, the swab 12 may be a standard off-the shelf swab.

In use, similar to the implementations described above, the swab 12 is used to collect a fluid sample. The cap 50 is screwed onto (or otherwise attached to) the receptacle with the swab 12 now contained therein. As the cap closes down on the receptacle, the “tube” end of the swab 12 (that is, end 27) pierces the seal/pouch 26 in the cap 50, releasing reagent 22. Reagent then flows through the tube, thru and past the swab 12, thru the opening 56 in the receptacle 52 then into the drying region 18. Once the fluid is introduced into the drying region, it reaches the paper 17 and desiccant 16.

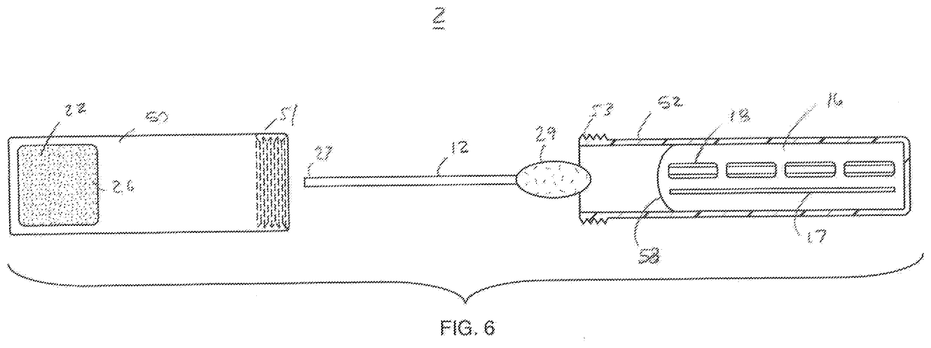

FIG. 6 is another embodiment with a screw-on cap 50, but having the drying region 16 disposed on an end of the receptacle 52 instead of alongside it. This permits the receptacle 52 to have a convenient cylindrical form for manufacture and handling. In this implementation, as the cap closes down on the receptacle, the “collection” end of the swab 12 (that is, end 27) again pierces the seal/pouch 26 in the cap 50, releasing reagent 22, The action of closing the cap 50 further breaches a seal 58 exposing the drying region. Reagent 22 then flows through past the swab 12, collecting the fluid sample and then continuing thru the breached seal 58 and into the drying region 18. Once the fluid is introduced into the drying region, it reaches the paper 17 and desiccant 16 as in the other embodiments.

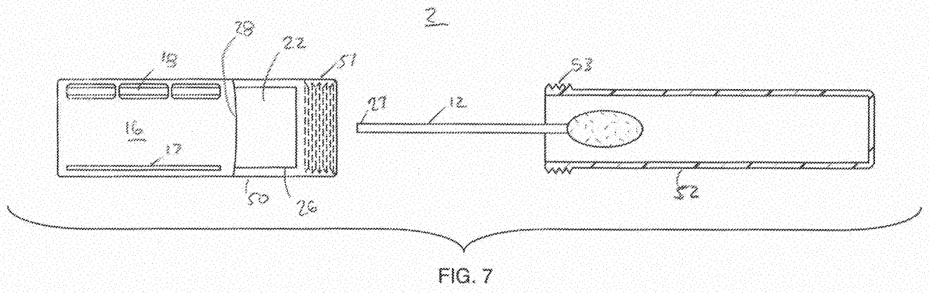

FIG. 7 is yet another configuration where the reagent 22 and drying region 16 are each enclosed in the cap 50. Here both the swab 12 and the media receptacle 52 may be standard off the shelf components, with only the cap 50 having special features. In this arrangement, the seal 28 is preferably disposed between the reagent 22 and the paper 17 and desiccant 18. In that way the drying region will be kept from ambient moisture and contamination until the cap 50 is pressed onto the receptacle.

In use of the FIG. 7 configuration, the swab 12 is placed in the standard media receptacle. The cap 50 has a pierce-able seal/pouch 26, which is pierced by the end 27 of the swab 27 as the cap 50 is screwed down. Once the cap 50 is screwed onto receptacle 52, the device 2 may be stood on end with the cap 50 on the bottom, encouraging liquid flow from the swab 12, mixing with the reagent 22 and passing into the drying region 16 with the paper 17 and desiccant 18.

Although threads are shown with the cap 50 having the outside thread (larger diameter) and the receptacle 52 having the inside thread (smaller diameter), these thread orientations can be reversed with the cap 50 having the inside thread (smaller diameter) and the receptacle 52 having the outside thread (larger diameter).

In some implementations, reagent may be contained in a pouch or by a seal that has pre-formed perforations that are more easily broken or ruptured as it comes into contact with the swab.

In some implementations, the cap may include an internal element that is caused to come into contact with the seal/pouch when the cap is screwed onto the receptacle.

A user-initiated mechanical force element, such as a knob, tab or plunger, may be accessible from outside the assembled cap/receptacle, that ruptures the pouch seal.

in another arrangement, a pull tab may be attached to the pouch/seal or other membrane enabling it to be ruptured before attaching the cap.

A secondary cap may be provided for cap 50 that is unscrewed to reveal the opening (and the paper/desiccant inside). In this arrangement, no membrane to seal may be necessary as the cap and the secondary cap would enclose the reagent until needed.

A container such as a bag or pouch may be provided for the cap 50, to keep the enclosed paper/desiccant sterile and dry until the point of use.

Variations or additions to the embodiments described above are possible. For example:

the biological sample may contain RNA;

the drying agent is a desiccant, an oxygen scrubber, or some combination thereof;

the cap may be designed to easily and securely attach the housing;

the cap may have a pierceable seal that is pierced when the cap is closed onto the housing;

the device may have other adaptations to collect samples for laboratory testing, on the spot testing;

the biological sample may be blood or a blood component;

the liquid reagent may contain a salt or a buffer;

the paper may be treated with sucrose or an anti-microbial;

the liquid reagent may be or may contain an anti-microbial;

the liquid reagent may contain bovine serum albumin;

the drying area may be blocked with bovine serum albumin;

the tube may contain a wicking element or agent or be composed of a wicking material;

the housing may have an o-ring to seal against the cap, or the cap may have an o-ring to seal against the housing;

the drying region may comprise or contain a paper substrate or an array of such substrates;

the drying region may be a wick being cylindrical, flat, or rectangular in nature;

the drying region may be an absorbent medium such as a desiccant tab or tabs;

the liquid reagent may be dispensed through the swab and sample by way of the tube connecting the swab to the interior body;

the sample and liquid reagent may be dispensed into the cap where it is absorbed by the drying region and the exposed to the drying agent;

the liquid reagent may be separated from the swab element by a pierceable membrane activated by pushing the housing into the cap; and/or

the liquid reagent may be separated from the swab element by a chamber that can only be accessed by twisting the cap with regards to housing.

While this invention has been particularly shown and described with references to example embodiments thereof, it will be understood by those skilled in the art that various changes in form and details may be made therein without departing from the scope of the invention encompassed by the appended claims.

What is claimed is:

1. A device for collecting and stabilizing a body fluid sample comprising: a housing comprising:
a swab element,
a tube connected to the swab element at one end, and to the interior body of the device at the other end
a drying region; and
a drying agent disposed in the drying region; and
a cap, adapted to removably engage the housing adjacent the swab element, the cap comprising:
a liquid reagent; and
a sealing element, to retain the reagent inside.

2. A device for collecting and stabilizing a biological sample comprising: a housing comprising:
a swab element,
a tube connecting the swab element to a handle having an interior region a drying region
a vent or series of vents to allow water vapor to escape
a cap, adapted to removably engage the housing adjacent to the swab element, the cap comprising:
a chamber containing a liquid reagent; and
a sealing element, to retain the reagent inside.

3. A device for collecting and stabilizing a biological sample comprising: a housing comprising two sections,
with a first section comprising:
a swab element,
a tube connecting the swab element to a handle having an interior region, a drying region, and
with a second section, adapted to engage the housing adjacent to the swab element when moved into place onto the housing, the second section further comprising:
a chamber containing a liquid reagent, and
a sealing element, to retain the reagent inside.

4. A device for collecting and transport a biological sample comprising: a housing comprising:
a swab element,
a tube connecting the swab element to a handle having an interior region a drying region
a transport medium within the dry region configured to absorb the sample a cap, adapted to engage the housing adjacent to the swab element, comprising:
a chamber containing a liquid reagent
a sealing element, to retain the reagent inside.

5. A device for collecting and stabilizing a biological sample comprising: a housing comprising:
a saliva collection element,
a tube connecting the saliva collection element to a handle having an interior region,
a drying region
a cap, adapted to engage the housing adjacent to the saliva collection element,
comprising: a chamber containing a liquid reagent
a sealing element, to retain the reagent inside.

6. A device for collecting and stabilizing a biological sample comprising: a housing comprising:
a swab element,
a tube connected to the swab element at one end, and to the interior body of the device at the other end
a liquid reagent,
a sealing element, to releasably retain the liquid reagent upon application of a mechanical force;
a drying region; and
a drying agent disposed in the drying region.

7. The device of claim 1 where the biological sample contains RNA.

8. The device of claim 1 where the drying agent is a desiccant.

9. The device of claim 1 where the drying agent is an oxygen scrubber.

10. The device of claim 1 where the drying agents are a combination of desiccant and oxygen scrubber.

11. The device of claim 1 where the cap is attached to the housing.

12. The device of claim 5 where the cap has a pierceable seal that is pierced when the cap is closed.

13. The device of claim 5 where the drying agent is one or more of a desiccant, an oxygen scrubber.

14. The device of claim 1 used to collect samples for laboratory testing.

15. The device of claim 1 used to collect samples for on the spot testing.

16. The device of claim 1 where the biological sample is blood or a blood component.

17. The device of claim 1 where the liquid reagent contains a salt.

18. The device of claim 1 where the liquid reagent contains a buffer.

19. The device of claim 1 where the paper is treated with sucrose.

20. The device of claim 1 where the liquid reagent contains an anti-microbial.

21. The device of claim 1 where the paper is treated with an anti-microbial.

22. The device of claim 1 where the tube contains a wicking element.

23. The device of claim 1 where the housing has an o-ring to seal against the cap.

24. The device of claim 1 where the cap has an o-ring, to seal against the housing.

25. The device of claim 1 in which the drying region is a paper substrate.

26. The device of claim 1 in which the drying region is an array of paper substrates.

27. The device of claim 1 in which the drying region is a wick being cylindrical, flat, or rectangular in nature.

28. The device of claim 1 in which the drying region is an absorbent medium.

29. The device of claim 1 in which the drying agent is a desiccant tab or tabs.

30. The device of claim 6 where the liquid reagent is dispensed through the swab and sample by way of the tube connecting the swab to the interior body.

31. The device of claim 6 where the sample and liquid reagent are dispensed into the cap where it is absorbed by the drying region and the exposed to the drying agent.

32. The device of claim 6 where the liquid reagent is separated from the swab element by a pierceable membrane activated by pushing the housing into the cap.

33. The device of claim 6 where the liquid reagent is separated from the swab element by a chamber that can only be accessed by twisting the cap with regards to housing.

34. A device for collecting and stabilizing a body fluid sample comprising: a housing comprising:
a swab element,
a container to receive the swab element, the container being in fluid communication with:
a drying region; and
a drying agent disposed in the drying region; and
a cap, adapted to removably engage the housing adjacent the swab element, the cap comprising:
a liquid reagent; and
a sealing element, to retain the reagent inside.

35. The device of claim 33 wherein the cap removably engages the housing via threads.

36. The device of claim 33 wherein the container is placed in fluid communication with the drying region when the cap engages the housing.

37. The device of claim 33 wherein an end of the swab element breaks the sealing element when the cap engages the housing.
